(19) United States
(12) Patent Application Publication
(10) Pub. No.: US 2012/0310066 A1
(43) Pub. Date: Dec. 6, 2012

(54) METHOD AND APPARATUS FOR CREATING A HIGH RESOLUTION MAP OF THE ELECTRICAL AND MECHANICAL PROPERTIES OF THE HEART

(75) Inventors: Yehoshua Shachar, Santa Monica, CA (US); Bruce Marx, Ojai, CA (US); Laszlo Farkas, Ojai, CA (US); David Johnson, West Hollywood, CA (US); Leslie Farkas, Ojai, CA (US)

(73) Assignee: MAGNETECS, INC., Inglewood, CA (US)

(21) Appl. No.: 13/491,300

(22) Filed: Jun. 7, 2012

Related U.S. Application Data
(63) Continuation of application No. 12/113,804, filed on May 1, 2008.

(51) Int. Cl. A61B 5/042; A61B 5/053; A61B 5/0402; A61M 25/095

(52) U.S. Cl. 600/374

(57) ABSTRACT

A system method that tracks one or more points on the surface of a cardiac tissue throughout a cardiac cycle and collect various types of data points which are then subsequently used to generate a corresponding model of the tissue and display the model as a 3D color coded image is described. In one embodiment, the system determines the position and orientation of a distal tip of a catheter, manipulates the catheter tip so as to maintain constant contact between the tip and a region of cardiac tissue using the impedance method, acquires positional and electrical data of the tip-tissue configuration through an entire heartbeat cycle, repeats the measurements as many times as needed in different tissue regions, and forms a 3D color coded map displaying various mechanical and electrical properties of the heart using the acquired data.

This application is a continuation of U.S. patent application Ser. No. 12/113,804, filed May 1, 2008, the entirety of which is incorporated herein by reference and is to be considered part of this specification.

### BACKGROUND OF THE INVENTION

1. Field of the Invention

The invention relates to the field of acquiring high-resolution clinical models of various properties of the heart using an invasive medical device and systems and methods for locating and tracking said invasive device.

2. Description of the Related Art

Catheters and other invasive medical devices have long been used to perform electrical mapping of the heart. Typically, the catheter is inserted through a vein or other artery and is guided into the heart. Localization and mapping software operating from an outside system record the electrical and positional information of the distal tip of the catheter as it is guided about the volume of the heart chamber. The chamber walls are determined as the limit of acquired data, and the electrical properties associated with the presumed surface of the heart are mapped onto the generated single surface shell.

The prior art has primarily been concerned with acquiring a set of “data frames” of the tissue or organ that is to be modeled. These “data frames” are usually taken by an ultrasound or an MRI device and then the images obtained from them are used to interpolate or calculate various properties of the heart such as tissue velocity and strain rate for a specified region.

While prior methods are not without their successes, they are not without some serious disadvantages and limitations as well. For example, the prior art methods that employ a “data frame” acquisition procedure can not accurately track a point on the surface of a cardiac wall during a heartbeat.

Another disadvantage found in the prior art is the amount of error associated with such techniques and methods. For example, prior art that uses electrical localization distorts the positional information and limits the accuracy of the mechanical data. Additionally, prior art that applies geometric data to a previously sensed MRI and CT data inherently introduces approximations and errors that distort the mechanical data and average out specific irregularities that are extremely important when mapping biological tissue. Instead of tracking the position of a single point on the tissue surface, the prior art simply averages the point's position as it travels through the cardiac cycle and displays it as a static location, thus decreasing its usefulness.

These and other problems are solved by a device and method that tracks one or more points on the surface of a cardiac tissue throughout a cardiac cycle and collect various types of data points which are then subsequently used to generate a corresponding model of the tissue and display the model as a 3D color coded image.

### SUMMARY OF THE INVENTION

In one embodiment, the system determines the position and orientation of a distal tip of a catheter, manipulates the catheter tip so as to maintain constant contact between the tip and a region of cardiac tissue using the impedance method, acquires positional and electrical data of the tip-tissue configuration through an entire heartbeat cycle, repeats the measurements as many times as needed in different tissue regions, and forms a 3D color coded map displaying various mechanical and electrical properties of the heart using the acquired data.

In one embodiment, a magnetically-tipped catheter is inserted into a heart while under the QRS timing sequence (e.g., while undergoing the systole/diastole cycle). A catheter guidance and control imaging (CGCI) system guides the catheter around the heart by the generation of a shaped magnetic lobe.

The actual position, or AP, of the distal tip of the catheter is determined within the heart from either a third-party localization system, or from the CGCI's own detection unit in small time increments to allow a relatively continuous view of the tip position which is traveling with the chamber wall. The position of the catheter tip is determined accurately without the need for data registration with known models. The CGCI catheter position detection unit determines the position and orientation of the magnetic tip by sensing the field about the tip using four hall-effect magnetic sensors. These sensors triangulate the position of the tip based on the strength of the magnetic field, and then determine the orientation of the tip by analyzing the magnetic field direction at each sensor with respect to the sensor-catheter tip direction.

Once the catheter tip has been detected, the CGCI controller adjusts the catheter tip from the actual position to the desired position in a closed loop control mode at a specific portion of the QRS cycle of the heartbeat. This maintains the reference position at one specific portion of the heartbeat cycle and allows it to travel freely with the tissue through the rest of the systole/diastole cycle.

The desired position, or DP, is a data acquisition point on or near the heart chamber wall. The CGCI impedance seeking unit specifies the DP on a tissue path trajectory to the tissue surface through the point of expected tissue contact. In other words, the catheter tip is guided in a straight line from the heart chamber center to the DP to locate the surface of the chamber by achieving tissue contact at a point closest to the DP.

Once the system has determined exactly where the tissue surface is located, the position of the catheter tip, as it moves along the chamber wall, is tracked while surface contact is continuously determined by impedance. The CGCI controller accomplishes this by following a tracking point, or TP, as the impedance seeking unit (ISU) moves the distal tip of the catheter down the tissue path until tissue contact is made and maintained continuously through the heartbeat cycle. The ISU also monitors the conductivity as a function of path stability and retracts the catheter tip when tissue contact is made off the path, or gives additional catheter length when the catheter is pointing at the path but cannot reach it. The degree of contact is determined by the amount of impedance seen at the tip.

Once tissue contact is made at the DP and is maintained, the position of the tip, the contact impedance, and the electrical ECG of the heart are all measured over several heartbeats to form a single tissue location's characteristic data set. This process is repeated for several adjacent tissue locations and as each location is sampled, their data is assembled to represent the electrical and mechanical activity of the heart over the entire heartbeat cycle. Each catheter tip position is measured over many heartbeat cycles to assure that the position and associated electrical information is accurate.

The acquired data for position and electrical properties are then correlated with a system time and the current QRS phase of the heartbeat as a global reference for data processing.

Acquired data from the same location and heartbeat phase are filtered and then used to produce a time dependent geometric map of the heart throughout the heartbeat phases.

The acquired data is then manipulated and used to display several maps of various properties of the heart which then are placed over the recently created geometric map. These maps include the electrical potential, surface impedance, lateral surface velocity, internal tissue contraction, and the internal tissue contraction velocity.

### BRIEF DESCRIPTION OF THE DRAWINGS

FIG. 1 is a system block diagram showing the imaging and synchronization subsystem, the impedance seeking subsystem, and its function in obtaining the actual position and specifying the desired position.

FIG. 2 is a schematic diagram relating the tracking point, tissue path, and desired position.

FIG. 3 is a schematic diagram of the data signals and data derived from the external ECG signal.

FIG. 4 is a block diagram of the impedance seeking subsystem and the associated data acquisition signals.

FIG. 5 is a schematic diagram of the test for cyclic motion of the catheter tip.

FIG. 6 is a detailed view of the initial conditions of a displacement envelope.

FIG. 7 is a graph of the weighting as a function of the distance of the vertex from the transform.

FIG. 8a is a series of illustrations depicting how the displacement envelopes affect the geometry of the constructed mesh images when the heart phase is at 60 and 120 degrees.

FIG. 8b is a series of illustrations depicting how the displacement envelopes affect the geometry of the constructed mesh images when the heart phase is at 180 and 240 degrees.

FIG. 8c is a series of illustrations depicting how the displacement envelopes affect the geometry of the constructed mesh images when the heart phase is at 300 and 360 degrees.

FIG. 9 is a schematic diagram of the geometric calculations used to interpolate a scalar field across the surface of the mesh.

FIG. 10 is a schematic diagram illustrating the construction of triangles from a plurality of sample point locations.

### DETAILED DESCRIPTION OF THE PREFERRED EMBODIMENT

In one embodiment, the apparatus and method described herein acquires and displays the electrical and mechanical properties of the heart. The invention accomplishes this by first detecting the distal tip of a catheter that has been inserted into a heart, guiding the catheter tip around the chamber of the heart towards a desired position, detecting when the catheter has made tissue contact with the heart chamber wall, and finally measuring of the QRS signal of the heart throughout several heartbeats. This process can be repeated several times in order to accurately measure the tissue velocity, strain, and conduction velocity across the surface of the heart. Once the appropriate amount of data has been collected, a set of data-rich meshes may be constructed for each phase of motion of the heart and then used to calculate and display the physical properties in a detailed clinical map.

Tissue velocity mapping requires linking multiple surface position and electrical readings to the current phase of the heart. This measurement and simultaneous analysis can be done in real-time if enough electrodes are available, or in non-real time by taking readings of position and electro-potential over two or more heartbeats and compiles them in a more detailed interactive clinical model.

Position detection, contact confirmation, electrode ECG and heartbeat phase are collected relatively contemporaneously. If there is delay in the signals due to filtering, the time-shift can be corrected in the final model. In a real-time display, compensation for time-shifts due to filtering can be done using predictive algorithms. Such predictive algorithms rely upon the previous beats. Irregularities between beats will result in electrical readings and mechanical displacements and velocities being somewhat out of sync with each other, and derived data between the two will be inaccurate on a scale corresponding to the irregularity.

To generate an analytical model, a single catheter can be used to accumulate points about the chamber over many heartbeats, or a basket or lasso may also be used to gather more points at once. A visual reference model can be displayed to indicate the progress of data collection, as well as any fiducial reference markers, catheters and 3D models.

For a real-time model, a sufficient number of electrodes are placed in contact with the surface, and the flexibility of the interstitial material or mesh should be sufficient that the electrode tracks the same point on the moving surface. Quantities computed based on surface point tracking such as tissue velocity and strain can be computed in real-time using such a catheter. If the electrodes do not track the same point on the surface, the color-coded map of the ECG will be available to map onto the surface mesh.

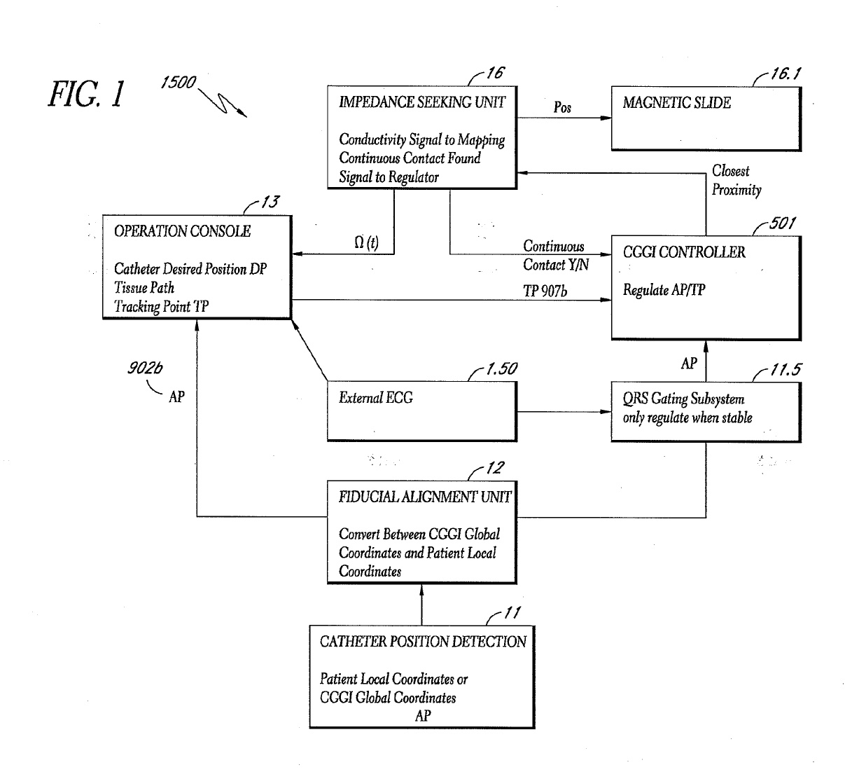

FIG. 1 is a system block diagram of the catheter guidance and control imaging (CGCI) system for position definition and guidance to locate and maintain tissue contact while acquiring data. The CGCI system for imaging and control of a catheter tip is described in U.S. patent application Ser. No. 11/697,690 “Method and Apparatus for Controlling Catheter Positioning and Orientation” and U.S. Pat. No. 7,280,863 “System and Method for Radar-Assisted Catheter Guidance and Control” and are hereby incorporated by reference.

Additionally, the current application is further supported and described in U.S. patent application Ser. No. 10/621,196 “Apparatus for Catheter, Guidance, Control, and Imaging,” U.S. patent application Ser. No. 11/331,781 “System and Method for Controlling Movement of a Surgical Tool,” U.S. patent application Ser. No. 11/331,994 “Apparatus and Method for Generating a Magnetic Field,” U.S. patent application Ser. No. 11/331,485 “System and Method for Magnetic Catheter tip,” U.S. patent application Ser. No. 11/140,475 “Apparatus and Method for Shaped Magnetic Field Control for Catheter, Guidance, Control and Imaging,” U.S. patent application Ser. No. 11/362,542 “Apparatus for Magnetically Deployable Catheter with Mosfet Sensors and Method for Mapping and Ablation,” and U.S. patent application Ser. No. 11/869,668 “System and Method for Radar-Assisted Catheter Guidance and Control” are all hereby incorporated by reference.

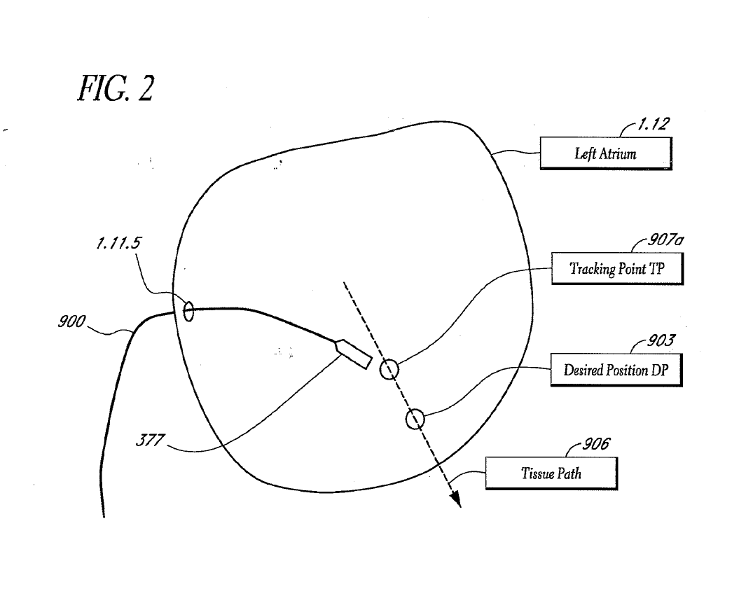

An actual position (AP) 902 and orientation of the distal end of the catheter is defined by external or internal subsystems of the CGCI system. Position detection can be globally referenced, or with respect to a six degree of freedom fiducial catheter, such as a custom coronary sinus catheter. Where the AP is defined with respect to the CGCI global coordinate system a fiducial alignment unit 12 maintains alignment with the patient's local coordinate system and converts between local and CGCI global coordinates. An operation console 13 in FIG. 1 defines the desired position (DP) 903 of the catheter tip (shown in FIG. 2) and a tissue path 906 (also shown in FIG. 2) that passes through the desired position. A CGCI controller 501 in FIG. 1 is given an initial tracking point TP 907a (shown in FIG. 2) on the tissue path 906 and magnetically steers the catheter to point to a tracking point TP 907a. The CGCI controller 501 sends the remaining positional error, the “closest proximity”, between the AP 902 and the TP 907a to an impedance seeking unit 16.

Contact confirmation with the surface of the heart chamber wall can be done by several methods, including, but not limited to, measuring surface conductivity. The impedance seeking unit 16 generates a tissue contact signal 16.2 based on the degree of tissue contact, namely, a small DC direct current is injected at each location of tissue contact and the conduction is measured. If the tip of the catheter is in contact with the surface, the conduction will be higher than if it was within the blood stream. The level of conductivity is recorded in the data set for future use. A minimum value can be set to limit data collection to good surface contact.

The impedance seeking unit 16 advances the catheter using a magnetic slide 16.1 until continuous tissue contact is found by monitoring the tissue contact signal 16.2, or until the point TP 907a is reached, or in other words, when the AP 902 equals the TP 907a. If the point TP 907a is reached before continuous contact is made, the CGCI controller 501 advances the point TP 907a in a positive direction along the tissue path 906 by a desired distance (e.g., 2 mm at a time), so as to maintain a predictable and repeatable approach to tissue contact. When full tissue contact is maintained, the impedance seeking unit 16 signals the CGCI controller 501 to stop all regulation and the catheter tip 377 is allowed to ride with the tissue surface under the current magnetic forces. If full tissue contact is made, but the location is too far from the tissue path (that is, the AP 902a is greater than a specified distance (e.g., 5 mm) from the TP 907a), the impedance seeking unit 16 retracts the catheter 900 a distance (e.g., 5 mm) to allow the CGCI controller 501 to redirect the catheter tip 377 towards the TP 907a before the impedance seeking unit 16 advances the catheter again.

The magnetic slide 16.1 is used to regulate to the tracking point (TP) 907b. If the tracking point TP 907b is acquired by the magnetic slide 16.1 and the tissue contact signal 16.2 shows incomplete contact, the physical tracking point TP 907a is moved down the tissue path 906 and the CGCI controller 501 regulates to the new tracking point, TP 907b.

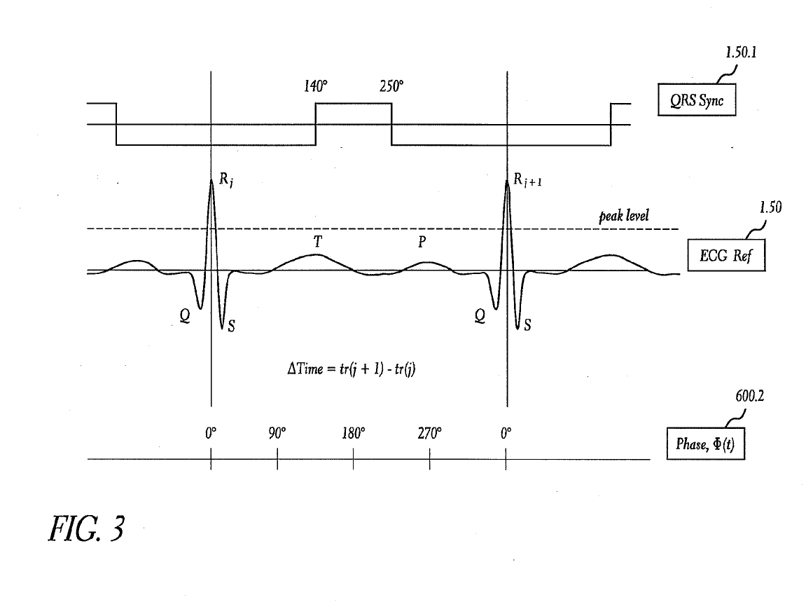

FIG. 2 is a schematic diagram of the left atrium 1.12 (facing the patient) and the tissue path 906 used by the CGCI system to guide the catheter to the moving tissue surface through the desired position DP 903 on the geometric static model. The CGCI system targets a unique position on the tissue surface that, while the tissue is moving, passes through or near the selected position on the static geometric model. The desired position, DP 903, is defined on or near the surface of the heart at a detected time on the QRS stable timing signal 1.50.1 (see FIG. 3). The tissue path 906 can be selectively defined as the surface normal of the geometric heart model at the point DP 903, a ray from the geometric center of the heart chamber through the DP 903, or a ray of any direction of expected tissue travel drawn through the point DP 903. The tracking point TP 907a is the closed-loop regulator target point sent to the CGCI controller 501. In FIG. 2, a catheter 900 is inserted into the left atrium 1.12, through the interatrial transseptum 1.11.5. The magnetic tip 377 is guided to the tracking point 907a by the CGCI controller 501. Once the catheter tip 377 reaches the tracking point TP 907a, the tracking point TP 907a is advanced down the tissue path 906 until the impedance signal 16.2 shows continuous tissue contact.

FIG. 3 is a schematic diagram of the signals and data derived from the external ECG signal. An External ECG signal 1.50, labeled ECG Ref, is used as a global reference signal to synchronize acquired data with a specific portion of the QRS heartbeat phase, φ(t) 600.2.

The heartbeat phase, φ(t) 600.2, is measured from R-peak to R-peak and is re-calculated after each heartbeat cycle to maintain the proper synchronization.

The QRS sync signal 1.50.1 provides a reference signal 10 to the CGCI controller 501 and CGCI operation console 13 when the heart is at its most stable point. This occurs between the end of the T-wave and the beginning of the P-wave, approximately 140-250° from the R-peak. In the QRS sync 1.50.1, the CGCI catheter position detection unit 11 measures the stable reference position of the catheter tip 377 and the CGCI controller may make position adjustments to the catheter to maintain the desired position DP 903a or tracking point, TP 907a.

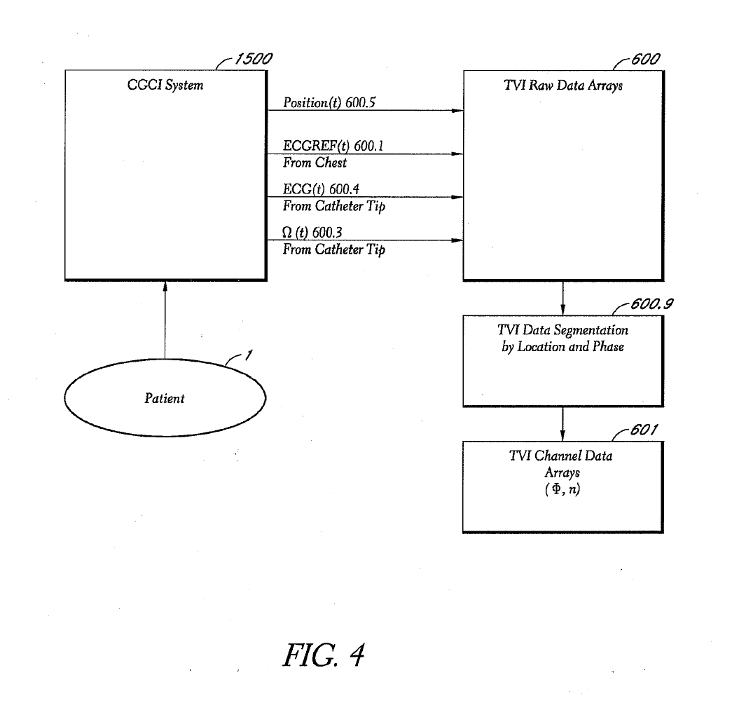

FIG. 4 is a schematic diagram of the tissue velocity imaging (TVI) data sets and processing routines. The CGCI system 1500 provides the external ECG reference signal ECGREF(t) 600.1, the internal catheter tip ECG signal ECG(t) 600.4, the catheter tip impedance signal Ω(t) 600.3, and the position of the catheter within the patient 1, position(t) 600.5.

Two data sets are created. The first data set contains records of each X, Y, Z position of the catheter tip 377, the ECG, the contact conductivity, and the system time. The second data set records the external ECG with respect to system time. The second set serves as an electrical fiducial reference frame for the data and can be used to reconstruct the electromechanical behavior over many regular or irregular heartbeats. System time will be replaced with heartbeat phase as a time reference in analytical models.

The ECG map can be appended with the heartbeat phase (φ) after the data is collected or while the data is being collected using a predictive algorithm. The phase is to be based on the period between the ECG's R intervals and the time since the last R peak.

The CGCI system 1500 measures the global external ECG reference signal from the patient's chest and produces the ECGREF(t) array 600.1, where t is the CGCI system 1500 operation time in milliseconds. The Position(t) array is produced from the CGCI system's 1500 positioning system and contains the xyz locations of the catheter tip 377. The tip electrocardial signal array, ECG(t) 600.4, and the tip impedance array, Ω(t) 600.3 are compiled from the sensed data at the catheter tip 377. These four arrays comprise the raw data arrays 600 which are used to create individual channel data arrays 601 for each location and phase.

The data segmentation routines 600.9 first calculates the heartbeat phase with respect to time, φ(t) 600.2, then separates out each mapped location's data into the individual channel data arrays 601 which are indexed by location number and heartbeat phase. These channel data arrays 601 contain both the electrical and mechanical properties of each tissue location which are then later used to form the analytical model.

After the data has been collected, it is processed into the DirectX multi-platform mesh format that not only contains the location vertex points of the heart wall, but also the electrical ECG values of those locations, the conductivity readings, and any other desired data. The data is then manipulated to form a 3D color coded mesh structure that contains sub-meshes for each phase of motion which are registered to each other such that their vertices represent the same point on the heart wall in order to provide a consistent and accurate set of geometric calculation points.

The external reference ECG signal is recorded in an array with respect to system time, ECGREF(t) 600.1, where t is defined in milliseconds. This array is used to create the array of heartbeat phase with respect to system time, φ(t) 600.2. This provides an index to relate all other system time referenced data acquisition signals to heartbeat phase.

ECGREF(t)→φ(t)

Heartbeat phase is used to sequence the data without respect to heart rate. When these data are assembled, the mechanical contraction of the heart along with the associated color ECG map may be displayed in either real-time or non-real-time models. Other derived data, such as the tissue contraction velocity and acceleration, or the lateral wall velocity and acceleration depend at least in part on a registration between each phase-model of the heart. Registration involves the marking of key points on the heart wall in each phase to accurately track all surface points between phases. Some displays use this registered model. An algorithm for automatically registering models use the number of points and fine movement between the phases. After registration, the mesh for each phase can be regenerated such that each mesh vertex point represents the same heart surface point.

The QRS signal R peaks are detected whenever the ECGREF signal rises above the peak detection level, and their system times are recorded in the tr(j) array, where j indicates heartbeat number.

ECGREF(t) has R peaks at times tr(0), tr(1), tr(2), . . . tr(j).

The phase at time t, which lies between successive heartbeat peaks at times tr(j) and tr(j+1) is derived by equation 1.

φ(t)=360*(t−tr(j))/(tr(j+1)−tr(j)) Eq. 1

Phase is given the integer values of 0 to 359 degrees. Values that round to 360 are given the phase value of 0.

The raw data arrays are then defined in Table 1 below.

TABLE 1
Definitions of Raw Data Arrays

| FIG. 4 reference number | Data Array Description | Data Array Name |
|---|---|---|
| Not shown | CGCI System Operation | T |
| | Time, ms | |
| 600.1 | External ECG Reference | ECGREF(t) |
| 600.2 | Phase at system time t | φ(t) |
| 600.3 | Impedance Signal, Ohms | Ω(t) |
| 600.4 | Catheter Tip ECG, Volts | ECG(t) |
| 600.5 | Tip Position XYZ | Position(t) |

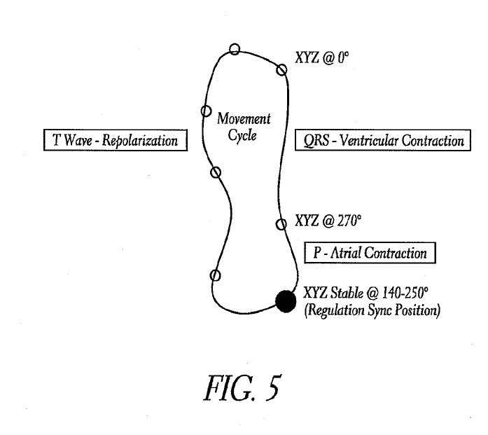

FIG. 5 is a schematic diagram depicting a continuous motion cycle of the catheter tip 377. The Position(t) data array is analyzed for segments of continuous tissue contact from the data recorded in the impedance array, Ω(t), and where the catheter Position(t) returns within short distance (e.g., 1 mm) of the home position at a phase of φ(t)=200°, indicating that the catheter tip travel conforms to a cyclic path. These continuous contact data segments are then mapped into the 360° phase-based arrays for that location, n. Where the array already contains data, the new data is averaged with the previous points. Where the array contains no data at the end of data compilation, the empty elements of the array are loaded with values that are interpolated from the nearest elements. This gives three arrays for each location, n, each containing 360 phase-mapped data values for the impedance, ECG and catheter tip 377 xyz position. These arrays and their respective symbols are displayed in Table 2 below.

TABLE 2
Mapped Arrays

| Data Array Description | Data Array Name |
|---|---|
| Heartbeat Phase, 0 to 359 degrees | φ |
| Impedance with respect to Phase φ, | Ω(φ, n) |
| Point n | |
| Local ECG as a function of Phase φ, | ECG(φ, n) |
| Point n | |
| Position of Catheter Tip for Phase φ, | Position(φ, n) |
| Point n | |

The electrical and mechanical data for each sampled location in the heart can now be directly accessed by heartbeat phase. From this data, color-coded maps can be created based the values at each point. A registered data set is required for properties that are relative to two points, or to view the change in value at the same physical location on the surface.

The base mesh consists of an array of Mc vertices each with the following structure displayed in Table 3:

TABLE 3
struct Left_Atrial_Vertex
{

| | | |
|---|---|---|
| VECTOR Pos; | | // Position |
| float | WEIGHT[Num_W]; | // Displacement Weighting |
| DWORD | TR_Index[Num_W]; | // Transform Index array |
| float | ECG; | // Potential |
| float | Imp; | // Impedance |
| DWORD | Color; | // Vertex Color |
| }; | | |

The base mesh steady state initial condition is φ(t)ₛₛ=140°, and the transform/displacement channels are represented by T(n)=DAT(φ, n)

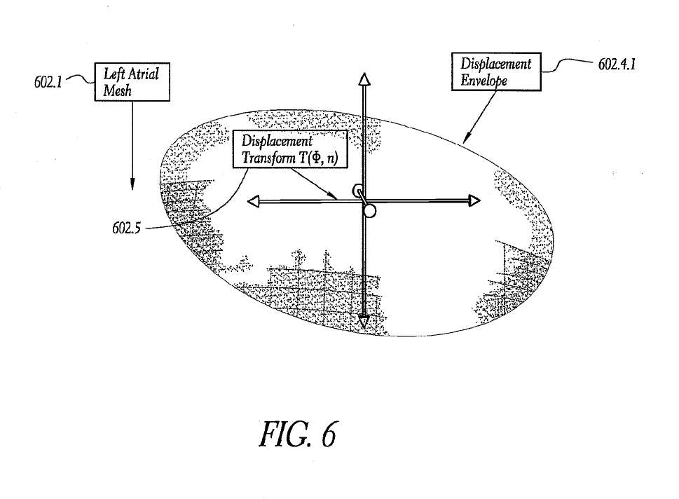

FIG. 6 is a detailed view of the initial conditions of a displacement envelope created by the invention. This process is performed one time after the dataset has been assembled.

Displacement envelopes are regions that affect the movement of the vertices that constitute the mesh as a function of the movement of the base displacement transform. The radius of the envelope is the distance to the nearest neighbors sample point.

Each vertex in the mesh is assigned weights and is associated with transforms of influence through the TR_Index array shown in Table 3 above. For each sample point Tₙ, the vertices of the mesh are processed. Weights are then calculated according to the following function including equation 2.

If (r>R), Weight(r)=0. Otherwise, x=r/R and

Weight(r)=1−(3x²−2x³), Eq. 2

where R≡E Radius of the left atrium 1.12 and r is a positive value.

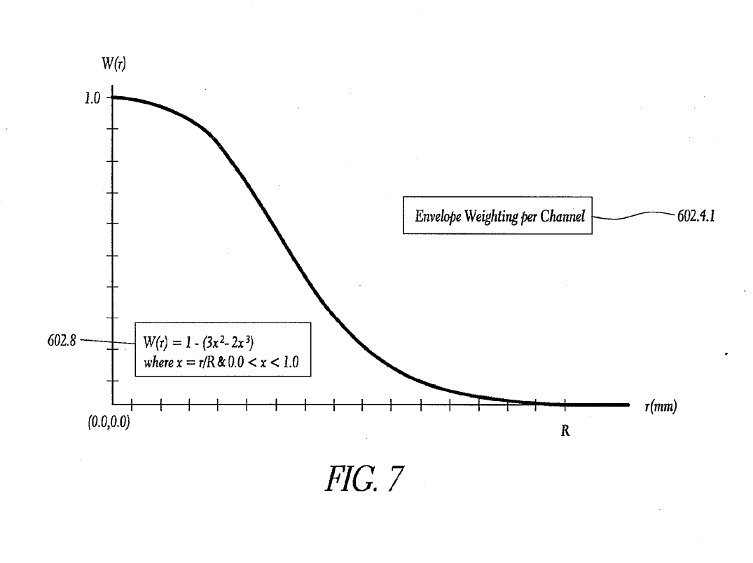

FIG. 7 shows the weighting as a function of the distance of the vertex from the transform Tₙ. The time domain representation of the left atrium is accomplished by computing the position of each vertex as a function of φ according to equation 3.

VertexPos=T₁·PW_{1+ . . .} Tₙ₋₁·PWₙ₋₁₊Tₙ·P(1.0−ΣWᵢ) where (i=1 . . . n) Eq. 3

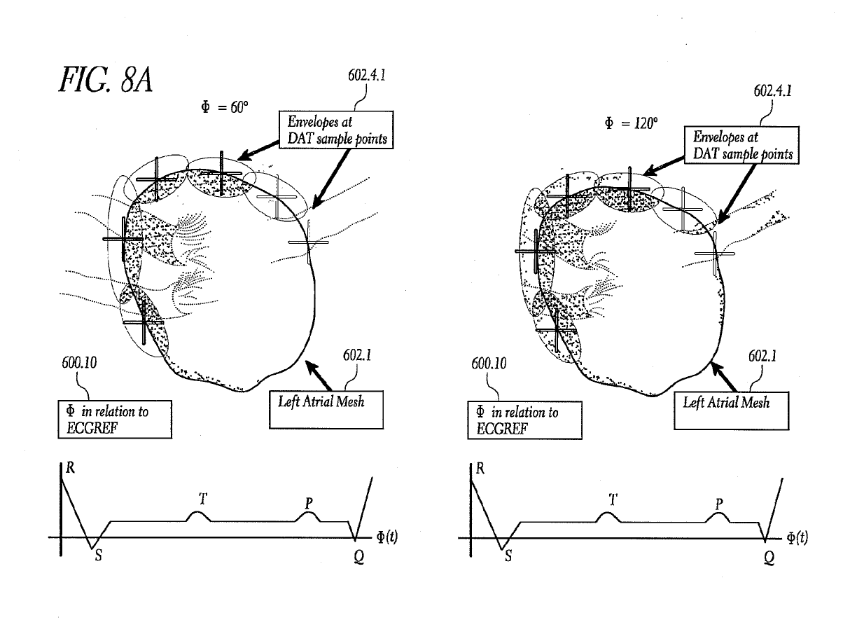

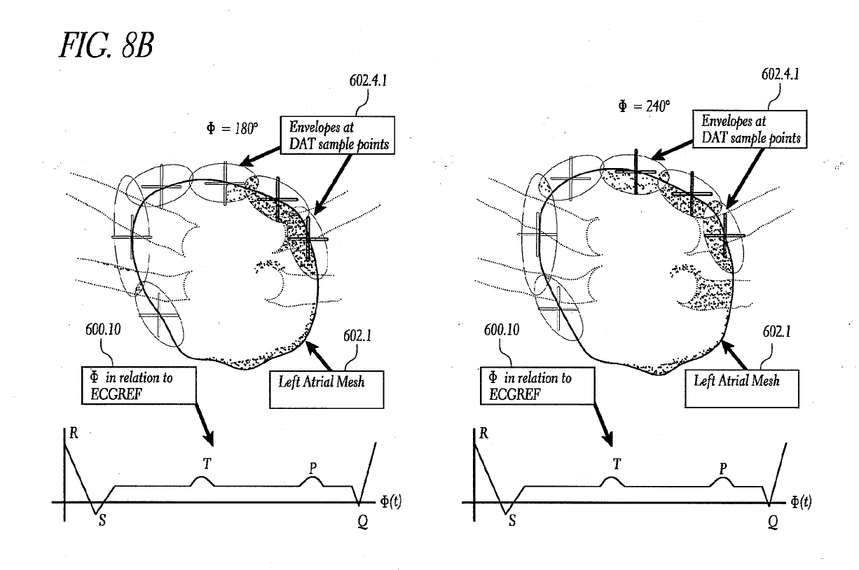

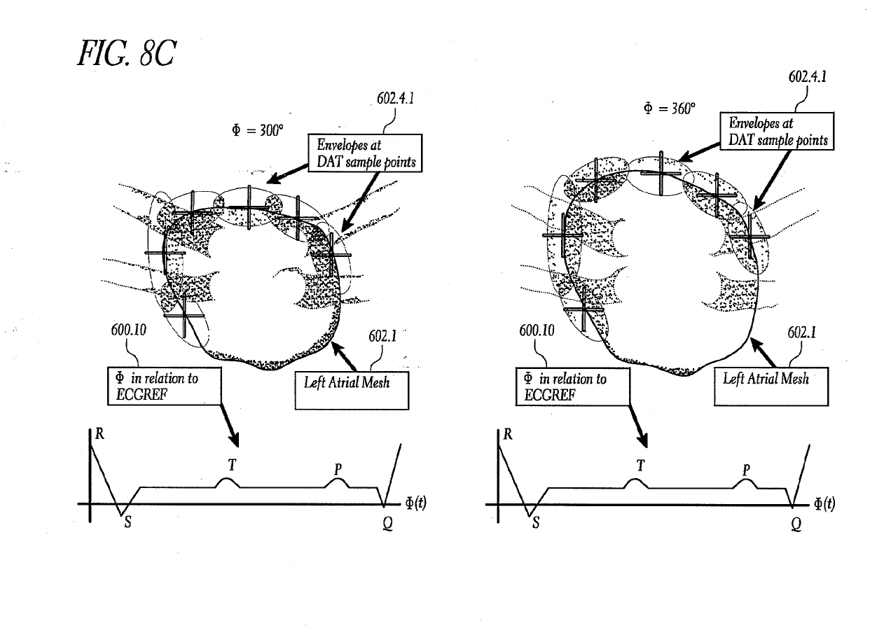

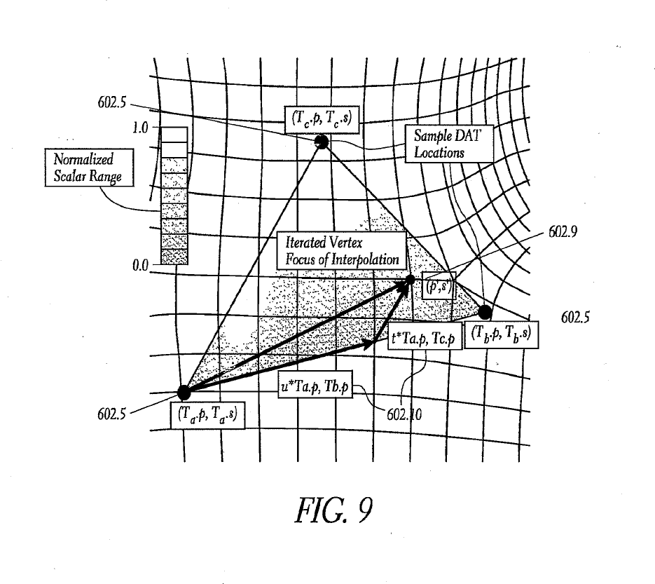

FIGS. 8a-8c are a series of illustrations depicting how the displacement envelopes affect the geometry of the constructed mesh images and reflect the mechanical properties of the left atrium. The two scalars recorded in Ω(φ, n) and ECG(φ, n) as shown in Table 3 above may be represented graphically on the left atrium 1.12 mesh using the following procedure. FIG. 9 illustrates the arrangement of the sample channels as points on a projection surface.

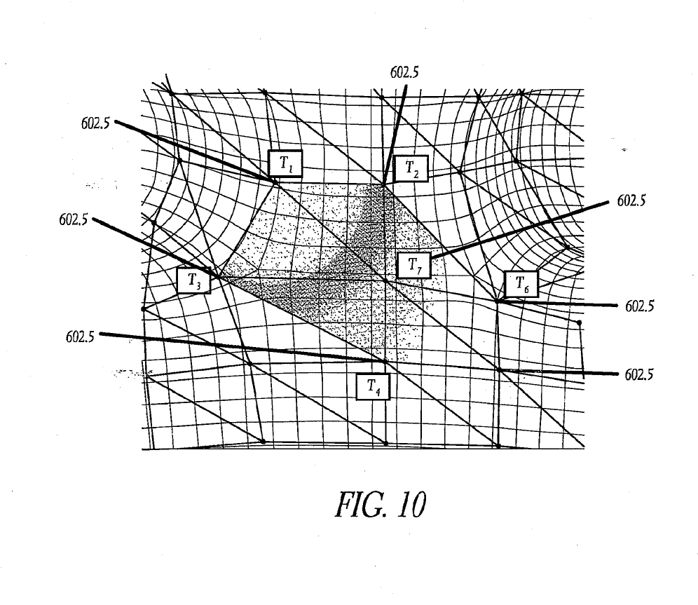

It is to be expressly understood that the procedure described below is conducted on each rendering frame for each triangle that constitutes the surface of the heart, not just for the six examples shown in FIGS. 8a-8c. Each vertex that makes up the left atrium mesh is considered. FIG. 10 depicts the calculation that occurs when a ray from the vertex of the triangle being processed intersects a ray along its normal surface. The point of intersection between these two rays is the focus of interpolation using equation 4 below.

s′=Tₐ·s+t(T_{c}·s−Tₐ·s)+u(T_{b}·s·Tₐ·s), Eq. 4

where t and u can be found using equations 5 and 6.

p′·x=Tₐ·p·x+t(T_{c}·p·x−Tₐ·p·x)+u(T_{b}·p·x−Tₐ·p·x) Eq. 5

p′·y=Tₐ·p·y+t(T_{c}·p·y−Tₐ·p·y)+u(T_{b}·p·y−Tₐ·p·y) Eq. 6

Once the appropriate amount of data has been collected and correctly weighted, the user of the apparatus then can use the data to determine a whole host of additional heart properties such as tissue displacement, contraction velocity, and contraction acceleration. These newly found properties can then be mapped on a position or phase basis using the electrical and mechanical differences between adjacent points. Data that is displayed is represented as change per degree of phase and may be linearly scaled to represent actual velocities in SI or English units for a given heart rate.

In cases where the real-time display of data is available, the 3D mesh formed by the electrode positions is displayed dynamically moving in space, or frozen and linked to a single phase of the heartbeat to better display derived data on its surface. Color-coded values for tissue contraction velocity, acceleration, electrical potential and conduction velocity are displayed dynamically on the surface of the mesh, and other derived values for electromechanical interaction would be available as well. Any shift in the location of the catheter tip 377 with respect to the surface will shift the view of the data with respect to that surface, and shifts in position of individual electrodes with respect to each other will momentarily affect the accuracy of the derived data. Filtering based on the collected data from previous heartbeats can be used to stabilize the display and increase the accuracy of the measurements in patients with predictable and repeating heart rhythms. Where the catheter tip 377 loses contact with the surface, the mesh will be missing that location and both the geometric model and derived color-coding will be more coarse in that region. Real-time data is collected and may be used later to reconstruct a more detailed analytical model.

For non-real-time models, data is collected over a number of locations and a number of heartbeats and assembled into a heartbeat phase-linked data set. For unregistered models, several color-coded data sets are displayed on the surface of the geometry, including the electrical map and conductivity map. Color-coded data sets are displayed on the associated phase of the heart geometry, and with some electrical-geometric inaccuracy, the color-coded maps are animated on the surface of a single phase of the heart's geometry. The animations can be played in a loop, and/or a slide bar can be used to jog the animation back and forth over the desired portion of the cycle.

Where a registration map has been created, the color-coded animations are more accurately displayed on a static geometric model. In addition, other data sets may be created and displayed. The knowledge of how a single point, or pairs of points move through the phases gives us the tissue velocity in the contracting tissue between the points, the lateral velocity of the wall, the associated accelerations, and a more accurate view of how the conduction velocity is occurring at a particular location. Other derived quantities that link the electrical and mechanical properties of the heart may be accurately mapped and used as clinical indicators. Two points can be placed on the model and the quantities between these points may be tracked in detail. This also allows the electrical and mechanical properties to be measured parallel or perpendicular to the muscle fiber direction.

The tissue displacement, σ, between location numbers n=i and n=j at phase φ is defined by equation 7:

σ(i,j,φ)=(ΔPi−ΔPj)/(Position(φ,i)−Position(φ,j)), Eq. 7

where

ΔPi=Position(φ,i)−Position(φ−1,i) Eq. 8

and

ΔPj=Position(φ,j)−Position(φ−1,j). Eq. 9

The relative contraction velocity between points on the surface, σ′, is defined by the first derivative of the strain a with respect to the phase change in equation 10.

σ′(i,j,φ)=σ(i,j,φ)−σ(i,j,φ−1) Eq. 10

Relative contraction acceleration between points on the surface is defined by the second derivative of the strain a with respect to the phase change in equation 11.

σ″=σ′(i,j,φ)−σ′(i,j,φ−1) Eq. 11

It is important to note that all surface quantities are displayed in normalized units per unit length.

The first derivative of Local ECG with respect to heartbeat phase is defined by equation 12:

ECG′(φ,i)=ECG(φ,i)−ECG(φ−1,i) Eq. 12

The global distance, velocity, and acceleration of location number i with respect to a fixed reference position, RefPos, are defined at a given phase by the equations 13, 14, and 15.

s(φ)=(Position(φ,i)−RefPos) Eq. 13

u(φ)=s(φ)−s(φ−1) Eq. 14

a(φ)=u(φ)−u(φ−1) Eq. 15

The tissue contraction versus electrical gradient is defined by equation 16:

TCE(i,j,φ)=σ(i,j,φ)/(ECG(φ,i)−ECG(φ,j)) Eq. 16

Additional displays contemplated by the applicant but not shown include the tissue contraction direction vectors of the heart superimposed over the previously constructed geometric surface map of the heart.

Many alterations and modifications may be made by those having ordinary skill in the art without departing from the spirit and scope of the invention. Therefore, it must be understood that the illustrated embodiment has been set forth only for the purposes of example and that it should not be taken as limiting the invention as defined by the following invention and its various embodiments.

Therefore, it must be understood that the illustrated embodiment has been set forth only for the purposes of example and that it should not be taken as limiting the invention as defined by the following claims. For example, notwithstanding the fact that the elements of a claim are set forth below in a certain combination, it must be expressly understood that the invention includes other combinations of fewer, more or different elements, which are disclosed in above even when not initially claimed in such combinations. A teaching that two elements are combined in a claimed combination is further to be understood as also allowing for a claimed combination in which the two elements are not combined with each other, but may be used alone or combined in other combinations. The excision of any disclosed element of the invention is explicitly contemplated as within the scope of the invention.

The words used in this specification to describe the invention and its various embodiments are to be understood not only in the sense of their commonly defined meanings, but to include by special definition in this specification structure, material or acts beyond the scope of the commonly defined meanings. Thus if an element can be understood in the context of this specification as including more than one meaning, then its use in a claim must be understood as being generic to all possible meanings supported by the specification and by the word itself.

The definitions of the words or elements of the following claims are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination.

Insubstantial changes from the claimed subject matter as viewed by a person with ordinary skill in the art, now known or later devised, are expressly contemplated as being equivalently within the scope of the claims. Therefore, obvious substitutions now or later known to one with ordinary skill in the art are defined to be within the scope of the defined elements.

The claims are thus to be understood to include what is specifically illustrated and described above, what is conceptionally equivalent, what can be obviously substituted and also what essentially incorporates the essential idea of the invention.

While the apparatus and method has or will be described for the sake of grammatical fluidity with functional explanations, it is to be expressly understood that the claims, unless expressly formulated under 35 USC 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 USC 112 are to be accorded full statutory equivalents under 35 USC 112. The invention can be better visualized by turning now to the following drawings wherein like elements are referenced by like numerals.

What is claimed is:

1. An apparatus for creating a high resolution map of the electrical and mechanical properties of the heart comprising:
a catheter;
a catheter guidance and control imaging system coupled to the catheter;
a data collection module for the catheter guidance and control imaging system to collect several different types of data from the catheter; and
a display for displaying the data collected by the catheter guidance and control imaging system in a three dimensional color-coded image format.

2. The apparatus of claim 1 wherein the catheter is magnetically tipped.

3. The apparatus of claim 2 wherein the catheter guidance and control imaging system further comprises a magnet system for generating a magnetic field in order to alter the course of the magnetically tipped catheter.

4. The apparatus of claim 3 wherein the catheter guidance and control imaging system alters the surrounding magnetic field for locating, orientating, and guiding the distal tip of the catheter to a desired position along an inner heart wall surface of a patient.

5. The apparatus of claim 4 wherein the catheter guidance and control imaging system maintains the distal tip of the catheter in a fixed position throughout a position of a cardiac cycle.

6. The apparatus of claim 5 further comprises a unit for measuring the impedance at the catheter tip and adjusting the distance from the catheter tip to the tissue surface so as to maintain a constant impedance reading.

7. The apparatus of claim 6 wherein the apparatus collects and records the system operation time, the ECG signal from the patient, the impedance signal from the catheter tip, the position of the tip, and the ECG signal from the catheter tip.

8. The apparatus of claim 7 wherein the data collected by the catheter guidance and control imaging system in a three dimensional color-coded image format further comprises correlating the obtained data with a measured heartbeat phase taken contemporaneously with the obtained data.

9. The apparatus of claim 8 wherein the format interpolates one or more data points that are found missing from the original data set.

10. The apparatus of claim 9 wherein the format calculates and displays tissue displacement between any two points located on the three dimensional image.

11. The apparatus of claim 9 wherein the means displays tissue contraction velocity between any two points located on the three dimensional image.

12. The apparatus of claim 9 wherein the apparatus displays tissue contraction acceleration between at least two points located on the three dimensional image.

13. The apparatus of claim 9 wherein the apparatus calculates and displays tissue displacement versus the electrical gradient between at least two points located on the three dimensional image.

14. A method of creating a high resolution map of the electrical and mechanical properties of the heart comprising:
determining the position and orientation of a tip of a catheter within a patient's heart using a catheter guidance and imaging system operatively coupled to the catheter;
changing the position of the catheter tip by altering the shape and polarity of a surrounding magnetic field;
guiding the distal tip to a desired position along the inner surface of the heart wall;
maintaining the catheter tip at the desired position along the inner surface of the heart wall for at least a portion of cardiac cycle;
acquiring first data based on the patient and the distal tip during a cardiac cycle;
calculating one or more electrical and mechanical properties of the heart from the first data;
processing the first data into a three dimensional color-coded image according to the various electrical and mechanical properties; and
displaying the three dimensional color-coded image on a display.

15. The method of claim 14 wherein maintaining the distal tip at the desired position along the inner surface of the heart wall for an entire cardiac cycle further comprises:
measuring the impedance value at the catheter tip; and
adjusting the distance from the catheter tip to the tissue surface so as to maintain a constant impedance reading.

16. The method of claim 15 wherein acquiring a said first data comprises:
recording the system operation time;
recording the ECG signal from the patient;
recording the impedance signal for the distal tip;
recording the position of the distal tip; and
recording the ECG signal from the distal tip.

17. The method of claim 16 wherein calculating said one or more electrical and mechanical properties of the heart from the obtained data further comprises correlating the obtained data with a measured heartbeat phase taken contemporaneously with the first data.

18. The method of claim 17 wherein calculating said one or more electrical and mechanical properties of the heart from the obtained data further comprises interpolating data points that are found missing from the original data set.

19. The method of claim 18 wherein calculating said one or more electrical and mechanical properties of the heart from the obtained data further comprises calculating tissue displacement between two previously measured points located on the inner surface of the heart wall.

20. The method of claim 18 wherein calculating said one or more electrical and mechanical properties of the heart from the obtained data further comprises calculating tissue contraction velocity between two previously measured points located on the inner surface of the heart wall.

21. The method of claim 18 wherein calculating said one or more electrical and mechanical properties of the heart from the obtained data further comprises calculating tissue contraction acceleration between two previously measured points located on the inner surface of the heart wall.

22. The method of claim 18 wherein calculating said one or more electrical and mechanical properties of the heart from the obtained data further comprises calculating tissue displacement versus the electrical gradient between two previously measured points located on the inner surface of the heart wall.

23. An apparatus for creating a high resolution map of the electrical and mechanical properties of the heart comprising:
a catheter;
a catheter guidance and control imaging system coupled to the catheter;
a means for the catheter guidance and control imaging system to collect several different types of data from the catheter; and
a means of displaying the data collected by the catheter guidance and control imaging system in a three dimensional color-coded image format.

24. The apparatus of claim 23 wherein the catheter is magnetically tipped.

25. The apparatus of claim 24 wherein the catheter guidance and control imaging system further comprises a means for generating a magnetic field in order to alter the course of the magnetically tipped catheter.

26. The apparatus of claim 25 wherein the catheter guidance and control imaging system further comprises a means for altering the surrounding magnetic field for locating, orientating, and guiding the distal tip of the catheter to a desired position along an inner heart wall surface of a patient.

27. The apparatus of claim 26 wherein the catheter guidance and control imaging system further comprises a means for maintaining the distal tip of the catheter in a fixed position throughout an entire cardiac cycle.

28. The apparatus of claim 27 wherein the means for maintaining the distal tip of the catheter in a fixed position throughout an entire cardiac cycle further comprises a means for measuring the impedance at the catheter tip and adjusting the distance from the catheter tip to the tissue surface so as to maintain a constant impedance reading.

29. The apparatus of claim 28 wherein the means for the catheter guidance and control imaging system to collect several different types of data from the catheter further comprises a means for collecting and recording the system operation time, the ECG signal from the patient, the impedance signal from the catheter tip, the position of the tip, and the ECG signal from the catheter tip.

30. The apparatus of claim 29 wherein the means of displaying data collected by the catheter guidance and control imaging system in a three dimensional color-coded image format further comprises a means of correlating the obtained data with a measured heartbeat phase taken contemporaneously with the obtained data.

31. The apparatus of claim 30 wherein the means of displaying data collected by the catheter guidance and control imaging system in a three dimensional color-coded image format further comprises a means for interpolating any data points that are found missing from the original data set.

32. The apparatus of claim 31 wherein the means of displaying data collected by the catheter guidance and control imaging system in a three dimensional color-coded image format further comprises a means for calculating and displaying tissue displacement between any two points located on the three dimensional image.

33. The apparatus of claim 31 wherein the means of displaying data collected by the catheter guidance and control imaging system in a three dimensional color-coded image format further comprises a means for calculating and displaying tissue contraction velocity between any two points located on the three dimensional image.

34. The apparatus of claim 31 wherein the means of displaying data collected by the catheter guidance and control imaging system in a three dimensional color-coded image format further comprises a means for calculating and displaying tissue contraction acceleration between any two points located on the three dimensional image.

35. The apparatus of claim 31 wherein the means of displaying data collected by the catheter guidance and control imaging system in a three dimensional color-coded image format further comprises a means for calculating and displaying tissue displacement versus the electrical gradient between any two points located on the three dimensional image.
